(19) United States
(12) Patent Application Publication
(10) Pub. No.: US 2026/0174588 A1
(43) Pub. Date: Jun. 25, 2026

(54) FLUID COLLECTION DEVICES, SYSTEMS, AND METHODS

(71) Applicant: PUREWICK CORPORATION, Covington, GA (US)

(72) Inventor: Jason Iain Glithero, McDonough, GA (US)

(21) Appl. No.: 19/539,961

(22) Filed: Feb. 13, 2026

Related U.S. Application Data
(63) Continuation of application No. 17/051,399, filed on Oct. 28, 2020, now Pat. No. 12,575,961.
(60) Provisional application No. 62/665,302, filed on May 1, 2018.

(51) Int. Cl. A61F 5/453; A61F 5/44; A61F 5/455; A61M 1/00

(52) U.S. Cl. CPC A61F 5/453; A61F 5/4401; A61F 5/4408; A61F 5/455; A61M 1/80; A61M 2202/0496; A61M 2210/1092; A61M 2210/1096

(57) ABSTRACT

Examples relate to systems, devices, and methods for removing fluid from a fluid collection device using a portable vacuum source operably coupled thereto. The fluid collection devices include urine collection devices shaped to complement the female or male anatomy near the respective urethras and the vacuum source is operably coupled to the fluid collection device via one or more sections of conduit.

### CROSS-REFERENCE TO RELATED APPLICATIONS

This application is a continuation of U.S. Application No. 17/051,399 filed on 28 October 2020, which is a U.S. Nationalization of PCT International Application No. PCT/US2019/029608 filed on 29 April 2019, which claims priority to U.S. Provisional Application No. 62/665,302 filed on 1 May 2018, the disclosure of each of which is incorporated herein in its entirety by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experience by pilots, drivers, and workers in hazardous areas. Additionally, fluid collection from the individual may be needed for monitoring purposes or clinical testing.

Bed pans and urinary catheters, such as a Foley catheter, can be used to address some of these circumstances. However, bed pans and urinary catheters have several problems associated therewith. For example, bed pans can be prone to discomfort, spills, and other hygiene issues. Urinary catheters be can be uncomfortable, painful, and can cause urinary tract infections.

Thus, users and manufacturers of fluid collection devices continue to seek new and improved devices, systems, and methods to collect urine.

### SUMMARY

Embodiments disclosed herein are related to devices, systems, and methods of using fluid collection devices. In an embodiment, a fluid collection system is disclosed. The fluid collection system includes a fluid storage container configured to hold a fluid. The fluid collection system includes a fluid collection device in fluid communication with the fluid storage container. The fluid collection device includes a fluid impermeable barrier at least partially defining a chamber, the fluid impermeable barrier also defining an opening extending therethrough, the opening configured to be positioned adjacent to a female urethra or have a male urethra positioned therethrough. The fluid collection device includes a conduit including an inlet and an outlet, the inlet being positioned within the fluid collection device and the outlet is configured to be in fluid communication with the fluid storage container. The fluid collection system includes a portable vacuum source in fluid communication with one or more of the fluid storage container or the fluid collection device, the portable vacuum source configured to draw fluid from the fluid collection device.

In an embodiment, a method to collect fluid is disclosed. The method includes positioning an opening of a fluid collection device adjacent to a female urethra or around a male urethra, the opening defined by a fluid impermeable barrier of the fluid collection device. The method includes receiving fluid from the female urethra or the male urethra into a chamber of the fluid collection device, the chamber of the fluid collection device at least partially defined by the fluid impermeable barrier. The method includes applying suction with a portable vacuum source effective to suction the fluid from the chamber via a conduit disposed therein and in fluid communication with the portable suction device.

In an embodiment, a fluid collection system is disclosed. The fluid collection system includes a fluid storage container configured to hold a fluid. The fluid collection system includes a fluid collection device in fluid communication with the fluid storage container. The fluid collection device includes a fluid impermeable barrier at least partially defining a chamber, the fluid impermeable barrier also defining an opening configured to be positioned adjacent to a female urethra or have a male urethra positioned therethrough. The fluid collection device includes a conduit including an inlet and an outlet, the outlet being in fluid communication with the fluid storage container and the inlet being positioned in a portion of the chamber selected to be at a gravimetrically low point of the fluid collection device when worn by a user. The fluid collection system includes a portable vacuum source in fluid communication with one or more of the fluid storage container or the fluid collection device via the conduit, the portable vacuum source configured to draw fluid from the fluid collection device via the conduit.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of a system for fluid collection, according to an embodiment.

FIGS. 2-4 are schematic cross-sectional views of female fluid collection devices, according to embodiments.

FIGS. 5-7 are schematic cross-sectional views of male fluid collection devices, according to embodiments.

FIG. 8 is a flow diagram of a method to collect fluid, according to an embodiment.

### DETAILED DESCRIPTION

Embodiments disclosed herein are related to devices, systems, and methods of using fluid collection devices and systems. The devices, systems, and methods of using fluid collection devices and systems include a portable vacuum source to remove urine from the fluid collection device. The portable vacuum source may allow for portable usage of the systems and methods herein such as in non-hospital environments.

In an example, a fluid collection device includes a fluid impermeable barrier that at least partially defines a chamber. The fluid impermeable barrier also defines an opening extending therethrough that is configured to be positioned adjacent to a female urethra or have a male urethra positioned therethrough. The fluid collection device also includes a tube having a channel extending between an inlet and outlet thereof. The inlet is configured to be coupled to a suction source and the outlet is configured to be in fluid communication with (e.g., fluidly coupled to) a fluid storage (vessel or container). The outlet is positioned downstream from the inlet. The channel also defines at least one aperture therein that fluidly couples an interior of the channel to the rest of the chamber.

The fluid collection devices disclosed herein are configured to collect fluid(s) from an individual. The fluid collected by the fluid collection devices can include urine. The fluid(s) collected by the fluid collection devices can also include at least one of vaginal discharge, penile discharge, reproductive fluids, blood, sweat, or other bodily fluids.

The fluid collection devices disclosed herein are configured to be used in fluid collection systems. The fluid collection systems disclosed herein include a gas source. Systems that include a gas source can, in some examples, resolve several problems associated with systems that include a vacuum source. For example, a system that includes a vacuum source draws fluid(s) towards the vacuum source and deposits most of the fluid(s) in a fluid storage container before the fluid(s) can reach the vacuum source. However, a small quantity of fluid(s) (e.g., vapor from the fluid(s)) can still reach the vacuum source, which can contaminate and/or damage (e.g., rust) the vacuum source. Additionally, a large quantity of the fluid(s) can reach the vacuum source when the fluid storage container is substantially full. However, a system that includes a gas source (e.g., compressed air) moves the fluid(s) away from the gas source, thereby preventing contamination and/or damage though splashing or spray of the fluid(s) may be a problem with a gas. For example, a gas source may be used to create a vacuum by flowing a gas past a connected end of the conduit at a perpendicular or oblique angle to the conduit to create a vacuum in the conduit. The fluids are pulled up the conduit and into the gas flow in the direction of the gas flow, which is away from the gas source. In another example, systems that include a vacuum source cannot be used in environments that do not include an available vacuum source (e.g., a patient’s room does not include a vacuum source or the vacuum source is being used). As such, systems that include a gas source can be used in environments that do not include an available vacuum source. A liquid source such as water can be used to create and implement a vacuum in the same way as the gas source. The vacuum source or gas source can be utilized with any of the devices or systems disclosed herein to remove a fluid therefrom.

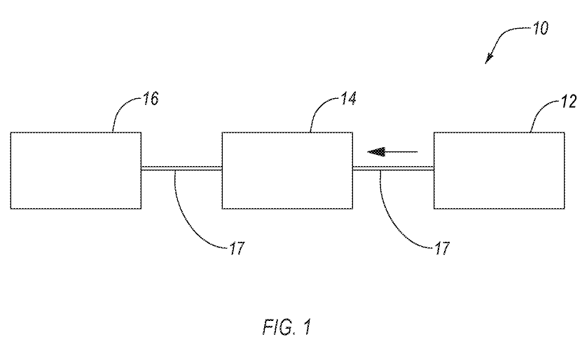

FIG. 1 is a block diagram of a system 10 for fluid collection, according to an embodiment. The system 10 includes a fluid collection device 12, a fluid storage container 14, and a portable vacuum source 16. The fluid collection device 12, the fluid storage container 14, and the portable vacuum source 16 may be fluidly coupled to (e.g., in fluid communication with) each other via one or more conduits 17. For example, fluid collection device 12 may be in fluid communication with one or more of the fluid storage container 14 or the portable vacuum source via the conduit 17. Fluid (e.g., urine or other bodily fluids) collected in the fluid collection device 12 may be removed from the fluid collection device 12 via the conduit 17 which protrudes into an interior region of the fluid collection device 12. For example, a first open end of the conduit 17 may extend into the fluid collection device 12 to a reservoir therein. The second open end of the conduit 17 may extend into the fluid storage container 14 or the portable vacuum source 16. The suction force may be introduced into the interior region of the fluid collection device 12 via the first open end of the conduit 17 responsive to a suction (e.g., vacuum) force applied at the second end of the conduit 17. The suction force may be applied to the second open end of the conduit 17 by the portable vacuum source 16 either directly or indirectly.

The suction force may be applied indirectly via the fluid storage container 14. For example, the second open end of the conduit 17 may be disposed within the fluid storage container 14 and an additional conduit 17 may extend from the fluid storage container 14 to the portable vacuum source 16. Accordingly, the portable vacuum source 16 may apply suction to the fluid collection device 12 via the fluid storage container 14. The suction force may be applied directly via the fluid storage container 14. For example, the second open end of the conduit 17 may be disposed within the portable vacuum source 16. An additional conduit 17 may extend from the portable vacuum source 16 to a point outside of the fluid collection device 12, such as to the fluid storage container. In such examples, the portable vacuum source 16 may be disposed between the fluid collection device 12 and the fluid storage container 14.

The fluid collection device 12 may be shaped and sized to be positioned adjacent to a female urethra or have a male urethra positioned therethrough (e.g., receive a penis therein). For example, the fluid collection device 12 may include a fluid impermeable barrier at least partially defining a chamber (e.g., interior region of the fluid collection device 12) of the fluid collection device. The fluid impermeable barrier also defines an opening extending therethrough from the external environment. The opening may be positioned adjacent to a female urethra or have a male urethra positioned therethrough. The fluid collection device 12 may include a fluid permeable membrane disposed within the fluid impermeable barrier. The fluid collection device 12 may include a fluid permeable support disposed within the fluid permeable membrane. The conduit 17 may extend into the fluid collection device 12 at a first end region, through one or more of the fluid impermeable barrier, fluid permeable membrane, or the fluid permeable support to a second end region of the fluid collection device 12. Exemplary fluid collection devices for use with the systems and methods herein are described in more detail below.

In some examples, the fluid storage container 14 may include a bag (e.g., drainage bag), a bottle or cup (e.g., collection jar), or any other enclosed container for storing bodily fluid(s) such as urine. In some examples, the conduit 17 may extend from the fluid collection device and attach to the fluid storage container 14 at a first point therein. An additional conduit 17 may attach to the fluid storage container 14 at a second point thereon and may extend and attach to the portable vacuum source 16. Accordingly, a vacuum (e.g., suction) may be drawn through fluid collection device 12 via the fluid storage container 14. Fluid, such as urine, may be drained from the fluid collection device 12 using the portable vacuum source 16.

In some examples, the portable vacuum source 16 may be disposed in or on the fluid collection device 12. In such examples, the conduit 17 may extend from the fluid collection device and attach to the portable vacuum source 16 at a first point therein. An additional conduit 17 may attach to the portable vacuum source 16 at a second point thereon and may extend out of the fluid collection device 12, and may attach to the fluid storage container 14. Accordingly, a vacuum (e.g., suction) may be drawn through fluid collection device 12 via the fluid storage container 14. Fluid, such as urine, may be drained from the fluid collection device 12 using the portable vacuum source 16. The portable vacuum source 16 may include one or more of a manual vacuum pump, and electric vacuum pump, a diaphragm pump, a centrifugal pump, a displacement pump, a magnetically driven pump, a peristaltic pump, or any pump configured to produce a vacuum. The portable vacuum source 16 may provide a vacuum or suction to remove fluid from the fluid collection device 12. In some examples, the portable vacuum source 16 may be powered by one or more of a power cord (e.g., connected to a power socket), one or more batteries, or even manual power (e.g., a hand operated vacuum pump). In some examples, the portable vacuum source 16 may be sized and shaped to fit outside of, on, or within the fluid collection device 12. For example, the portable vacuum source 16 may include one or more miniaturized pumps or one or more micro pumps. The portable vacuum sources 16 disclosed herein may include one or more of a switch, a button, a plug, a remote, or any other device suitable to activate the portable vacuum source. It should be understood that the portable vacuum sources 16 disclosed herein may provide a portable means of providing a suction or vacuum that allows use of the devices and systems herein outside of hospital or care facility environments where vacuum lines are plumbed into patient rooms or large (e.g., larger or heavier than a patient can readily carry) vacuum sources are located. For example, a portable vacuum source may be small and light enough to be carried by a user (e.g., patient) or aid (e.g., nurse) during transportation of the user.

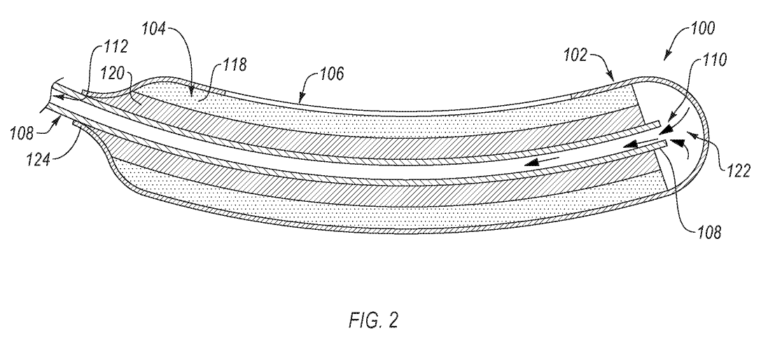

FIG. 2 is a schematic cross-sectional view of a fluid collection device 100, according to an embodiment. The fluid collection device 100 is an example of a female fluid collection device 100 that is configured to receive fluid(s) from a female. The fluid collection device 100 includes a fluid impermeable barrier 102. The fluid impermeable barrier 102 at least partially defines a chamber 104 (e.g., interior region) and an opening 106. The opening 106 is formed in and extends through the fluid impermeable barrier 102, thereby enabling fluid(s) to enter the chamber 104 from outside of the fluid collection device 100. The opening 106 can be configured to be positioned adjacent to a female urethra. The fluid collection device 100 also includes conduit 108 that is at least partially disposed in the chamber 104. The conduit 108 (e.g., a tube) includes an inlet 110 at a first end region and an outlet 112 at a second end region positioned downstream from the inlet 110. The conduit 108 fluidly couples an interior region of the chamber 104 with the fluid storage container (not shown) or the portable vacuum source (not shown).

In the illustrated embodiment, the conduit 108 is at least partially disposed in the chamber 104. For example, the conduit 108 may extend into the fluid impermeable barrier 102 from the first end region (e.g., proximate to the outlet 112) and may extend to the second end region (e.g., opposite the first end region) to a point proximate to a reservoir 122 such that the inlet 110 is in fluid communication with the reservoir 122. For example, in the illustrated embodiment, the inlet 110 is positioned in the reservoir 122. However, in other examples, the inlet 110 may be positioned flush with or behind an end of the fluid permeable membrane 118 that partially defines the reservoir 122. In some examples (not shown), the conduit 108 may enter the second end region and the inlet 110 of the conduit 108 may be disposed in the second end region (e.g., in the reservoir 122). The fluid collected in the fluid collection device 100 may be removed from the interior region of the chamber 104 via the conduit 108. The conduit 108 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some examples, the conduit 108 may include silicon or latex. In some examples, the conduit 108 may include one or more portions that are resilient, such as to by having one or more of a diameter or wall thickness that allows the conduit to be flexible.

The fluid collection device 100 may be positioned proximate to the female urethra and urine may enter the interior region of the fluid collection device 100 via the opening 106. The fluid collection device 100 is configured to receive the fluid(s) into the chamber 104 via the opening 106. For example, the opening 106 can exhibit an elongated shape that is configured to extend from a first location below the urethral opening (e.g., at or near the anus or the vaginal opening) to a second location above the urethral opening (e.g., at or near the clitoris or the pubic hair). The opening 106 can exhibit an elongated shape since the space between the legs of a female is relatively small when the legs of the female are closed, thereby only permitting the flow of the fluid(s) along a path that corresponds to the elongated shape of the opening 106 (e.g., longitudinally extending opening). The longitudinal axis or dimension of the fluid collection devices disclosed herein refers to the axis or dimension that is parallel to largest dimension of the device, such as axially along a cylindrical device as show in FIG. 2. The opening 106 in the fluid impermeable barrier 102 can exhibit a width that is measured transverse to the longitudinal direction of the fluid collection device 100 and may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. The opening 106 can exhibit a width that is greater than 50% of the circumference of the fluid collection device 100 since the vacuum (e.g., suction) through the conduit 108 pulls the fluid into the conduit 108. In some examples, the opening 106 may be vertically oriented (e.g., having a major axis parallel to the longitudinal axis of the device 100). In some examples (not shown), the opening 106 may be horizontally oriented (e.g., having a major axis perpendicular to the longitudinal axis of the device 100). In an example, the fluid impermeable barrier 102 can be configured to be attached to the individual, such as adhesively attached (e.g., with a hydrogel adhesive) to the individual. According to an example, a suitable adhesive is a hydrogel layer, such as those disclosed in U.S. Patent Application Publication No. 2017/0189225, the disclosure of which is incorporated herein by reference in its entirety.

The fluid impermeable barrier 102 may also temporarily store the fluid(s in the chamber 104. For example, the fluid impermeable barrier 102 can be formed of any suitable fluid impermeable materials, such as a fluid impermeable polymer (e.g., silicone, polypropylene, polyethylene, polyethylene terephthalate, a polycarbonate, etc.), a metal film, another suitable material, or combinations thereof. As such, the fluid impermeable barrier 102 substantially prevents the fluid(s) from exiting the portions of the chamber 104 that are spaced from the opening 106. The fluid impermeable barrier 102 can store fluid(s) in a reservoir 122 therein. The reservoir 122 may be disposed in any portion of the interior region of the chamber 104. For example, the fluid reservoir 122 may be positioned in the second end region of the chamber 104. In an example, the fluid impermeable barrier 102 can be air permeable and fluid impermeable. In such an example, the fluid impermeable barrier 102 can be formed of a hydrophobic material that defines a plurality of pores. In an example, at least one or more portions of at least an outer surface of the fluid impermeable barrier 102 can be formed from a soft and/or smooth material, thereby reducing chaffing. The fluid impermeable barrier 102 may include markings thereon, such as one or more markings to aid a user in aligning the device 100 on the wearer. For example, a line on the fluid impermeable barrier 102 (e.g., opposite the opening 106) may allow a healthcare professional to align the opening 106 over the urethra of the wearer. In examples, the markings may include one or more of alignment guide or an orientation indicator, such as a stripe or hashes. Such markings may be positioned to align the device 100 to one or more anatomical features such as a pubic bone, etc.

The fluid collection device 100 can include a fluid permeable membrane 118 disposed in the chamber 104. The fluid permeable membrane 118 can cover at least a portion (e.g., all) of the opening 106. The fluid permeable membrane 118 can be configured to wick any fluid away from the opening 106, thereby preventing the fluid from escaping the chamber 104. The permeable properties referred to herein can be wicking, capillary action, diffusion, or other similar properties or processes, and are referred to herein as “permeable” and/or “wicking.” Such “wicking” may not include absorption into the wicking material. The fluid permeable membrane 118 can also wick the fluid generally towards an interior of the chamber 104, as discussed in more detail below. The fluid permeable membrane 118 can include any material that can wick the fluid. For example, the fluid permeable membrane 118 can include fabric, such as a gauze (e.g., a silk, linen, polymer based materials such as polyester, or cotton gauze), another soft fabric (e.g., jersey knit fabric or the like), or another smooth fabric (e.g., rayon, satin, or the like). Forming the fluid permeable membrane 118 from gauze, soft fabric, and/or smooth fabric can reduce chaffing caused by the fluid collection device 100.

The fluid collection device 100 can include a fluid permeable support 120 disposed in the chamber 104. The fluid permeable support 120 is configured to support the fluid permeable membrane 118 since the fluid permeable membrane 118 can be formed from a foldable, flimsy, or otherwise easily deformable material. For example, the fluid permeable support 120 can be positioned such that the fluid permeable membrane 118 is disposed between the fluid permeable support 120 and the fluid impermeable barrier 102. As such, the fluid permeable support 120 can support and maintain the position of the fluid permeable membrane 118. The fluid permeable support 120 can include any material that can wick the fluid. The fluid permeable support 120 can be formed from any fluid permeable material that is less deformable than the fluid permeable membrane 118. For example, the fluid permeable support 120 can include a porous nylon structure. In an example, the fluid permeable support 120 can be omitted from the fluid collection device 100.

In an example, the fluid permeable membrane 118 and the fluid permeable support 120 can at least substantially completely fill the portions of the chamber 104 that are not occupied by the conduit 108. In another example, the fluid permeable membrane 118 and the fluid permeable support 120 may not substantially completely fill the portions of the chamber 104 that are not occupied by the conduit 108. In such an example, the fluid collection device 100 includes the reservoir 122 disposed in the chamber 104. The reservoir 122 is a substantially unoccupied portion of the chamber 104. The reservoir may be defined between the fluid impermeable barrier 102 and one or both of the fluid permeable membrane 118 and the fluid permeable support. The fluid(s) that are in the chamber 104 can flow through the fluid permeable membrane 118 and/or fluid permeable support 120 to the reservoir 122. The reservoir 122 can store at least some of the fluid(s) therein.

In an example, the reservoir 122 can be located at the portion of the chamber 104 that is closest to the inlet 110 (e.g., the second end region). However, the reservoir 122 can be located at different locations in the chamber 104. For example, the reservoir 122 can be located at the end of the chamber 104 that is closest to the outlet 112. In another example, the fluid collection device 100 can include multiple reservoirs, such as a first reservoir that is located at the portion of the chamber of the chamber 104 that is closest to the inlet 110 (e.g., second end region) and a second reservoir that is located at the portion of the of the chamber 104 that is closest to the outlet 112 (e.g., first end region). In another example, the fluid permeable support 120 is spaced from at least a portion of the conduit 108 and the reservoir 122 can be the space between the fluid permeable support 120 and the conduit 108.

In some examples, the fluid collection device 100 may be substantially cylindrical, ellipsoid, prismatic, or any other shape suitable for complementing or contouring to the vaginal region of a female subject. The cross-sectional shape of the fluid collection devices disclosed herein may include any of various shapes or sizes. For example, the cross-sectional shape (transverse to the longitudinal axis) may be substantially round (e.g., circular), elliptical, rectangular, triangular, irregular (e.g., having no specific shape), etc.

Other embodiments of fluid impermeable barriers, fluid permeable membranes, fluid permeable supports, chambers, and their shapes and configurations are disclosed in U.S. Patent Application No. 15/612,325 filed on June 2, 2017; U.S. Patent Application No. 15/260,103 filed on September 8, 2016; and U.S. Patent Application No. 15/611,587 filed on June 1, 2017, the disclosure of each of which is incorporated herein, in its entirety, by this reference.

The fluid impermeable barrier 102, the fluid permeable membrane 118 and the fluid permeable support 120 can be configured to have the conduit 108 at least partially disposed in the chamber 104. For example, at least one of the fluid permeable membrane 118 and the fluid permeable support 120 can be configured to form a space that accommodates the conduit 108. In another example, the fluid impermeable barrier 102 can define an aperture 124 sized to receive the conduit 108 (e.g., at least one tube). The at least one conduit 108 can be disposed in the chamber 104 via the aperture 124. The apertures 124 can be configured to form an at least substantially fluid tight seal against the conduit 108 or the at least one tube thereby substantially preventing the fluid(s) from escaping the chamber 104. The fluid collected in the fluid collection device 100 may be removed from the interior region of the chamber 104 via the conduit 108. As shown in FIG. 2, the end of the conduit 108 may extend beyond the fluid permeable membrane 118 and/or fluid permeable support 120, such as into the reservoir 122. In some examples, the inlet 110 may not extend into the reservoir 122. In such examples, the inlet 110 may be disposed within the wicking material (fluid permeable membrane 118 and/or fluid permeable support 120) or a terminal end thereof. For example, an end of the conduit 108 may be coextensive with or recessed within the fluid permeable membrane 118 and/or fluid permeable support 120.

As previously discussed, the conduit 108 is configured to be coupled to and at least partially extend between one or more of the fluid storage container (not shown) and the portable vacuum source (not shown). In an example, the conduit 108 is configured to be directly connected to the portable vacuum source (not shown). In such an example, the conduit 108 can extend from the fluid impermeable barrier 102 by at least one foot, at least two feet, at least three feet, or at least six feet. In another example, the conduit 108 is configured to be indirectly connected to at least one of the fluid storage container (not shown) and the portable vacuum source (not shown). In some examples, the conduit is secured to a wearer’s skin with a catheter securement device, such as a STATLOCK® catheter securement device available from C. R. Bard, Inc., including but not limited to those disclosed in U.S. Patent Nos. 6,117,163; 6,123,398; and 8,211,063, the disclosures of which are all incorporated herein by reference in their entirety.

The inlet 110 and the outlet 112 are configured to fluidly couple (e.g., directly or indirectly) the portable vacuum source (not shown) to the chamber 104 (e.g., the reservoir 122). In an example, the inlet 110 and/or the outlet 112 can form a male connector. In another example, the inlet 110 and/or the outlet 112 can form a female connector. In an example, the inlet 110 and/or the outlet 112 can include ribs that are configured to facilitate secure couplings. In an example, the inlet 110 and/or the outlet 112 can form a tapered shape. In an example, the inlet 110 and/or the outlet 112 can include a rigid or flexible material.

Locating the inlet 110 at or near a gravimetrically low point of the chamber 104 enables the conduit to receive more of the fluid(s) than if inlet 110 was located elsewhere and reduce the likelihood of pooling (e.g., pooling of the fluid(s) can cause microbe growth and foul odors). For instance, the fluid(s) in the fluid permeable membrane 118 and the fluid permeable support 120 can flow in any direction due to capillary forces. However, the fluid(s) may exhibit a preference to flow in the direction of gravity, especially when at least a portion of the fluid permeable membrane 118 and/or the fluid permeable support 120 is saturated with the fluid(s). Accordingly, one or more of the inlet 110 or the reservoir 122 may be located in the fluid collection device in a position expected to be the gravimetrically low point in the fluid collection device when worn by a user.

As the portable vacuum source (FIG. 1) applies a vacuum/suction in the conduit 108, the fluid(s) in the chamber 104 (e.g., at the second end region such as in the reservoir 122) may be drawn into the inlet 110 and out of the fluid collection device 100 via the conduit 108. In some examples, the conduit may be frosted or opaque (e.g., black) to obscure visibility of the fluid(s) therein.

In an example, the conduit 108 is configured to be at least insertable into the chamber 104. In such an example, the conduit 108 can include one or more markers (not shown) on an exterior thereof that are configure to facilitate insertion of the conduit 108 into the chamber 104. For example, the conduit 108 can include one or more markings thereon that are configured to prevent over or under insertion of the conduit 108, such as when the conduit 108 defines an inlet 110 that is configured to be disposed in or adjacent to the reservoir 122. In another example, the conduit 108 can include one or more markings thereon that are configured to facilitate correct rotation of the conduit 108 relative to the chamber 104. In an example, the one or more markings can include a line, a dot, a sticker, or any other suitable marking.

In an example, one or more components of the fluid collection device 100 can include an antimicrobial material, such as an antibacterial material where the fluid collection device may contact the wearer or the bodily fluid of the wearer. The antimicrobial material can include an antimicrobial coating, such as a nitrofurazone or silver coating. The antimicrobial material can inhibit microbial growth, such as microbial growth due to pooling or stagnation of the fluid(s). In an example, one or more components (e.g., impermeable barrier 102, conduit 108, etc.) of the fluid collection device 100 can include an odor blocking or absorbing material such as a cyclodextrine containing material or a thermoplastic elastomer (TPE) polymer.

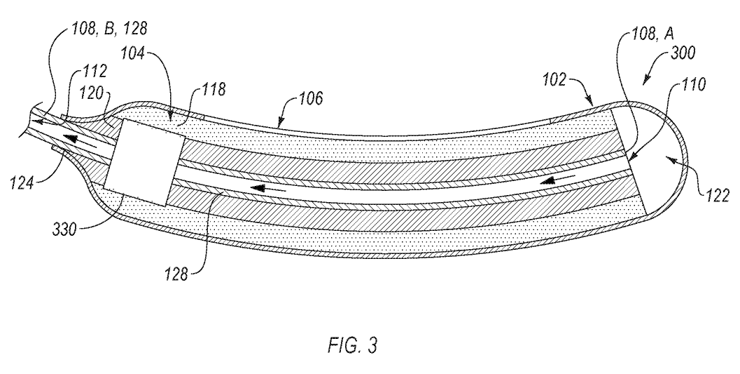

FIG. 3 is a schematic cross-sectional view of a fluid collection device 300, according to an embodiment. The fluid collection device 300 may include the portable vacuum source 330 disposed therein. Except as otherwise disclosed herein, the fluid collection device 300 can be the same as or substantially similar to the fluid collection device 100 of FIG. 2, in one or more aspects. For example, the fluid collection device 300 can include the fluid impermeable barrier 102 that defines the chamber 104 and the opening 106. The fluid collection device 300 also includes at least one of the fluid permeable membrane 118, the fluid permeable support 120, and the reservoir 122 disposed in the chamber 104.

The fluid collection device 300 includes the conduit 108 that is at least partially disposed in the chamber 104. The conduit 108 can include one or more walls that define an inlet 110 and the outlet 112. The inlet 110 enables at least some of the fluids that are present in the chamber 104 to enter the conduit 108. In an example, the conduit 108 can be configured to have the inlet 110 located at, near, or spaced at a gravimetrically low point of the chamber 104. In an example, the conduit 108 can be configured to have the at least one inlet 110 disposed in or adjacent to the reservoir 122.

The conduit 108 can be in fluid communication with the interior region of the chamber 104 via the fluid impermeable barrier 102. As such, the fluid impermeable barrier 102 can define an aperture 124. In an example, as illustrated, the aperture 124 enables the conduit 108 to extend outwardly from the chamber 104 when the conduit 108 is only partially disposed in the chamber 104. In some examples, the conduit 108 may include a plurality of separate sections. For example and as shown, the conduit 108 may include a first section A and a second section B. The first section A may include the inlet 110 extending from the distal end (e.g., first end region) to the portable vacuum source 330 and the B section may extend from the portable vacuum source 330 out of the aperture 124, such as to a fluid storage container (not shown).

The portable vacuum source 330 may include a pump, such as any of the portable vacuum pumps disclosed herein. For example, the portable vacuum source 330 may include a manual vacuum pump, a diaphragm pump, a centrifugal pump, a displacement pump, a magnetically driven pump, a peristaltic pump, or any pump configured to produce suction, a vacuum, or otherwise move a fluid. The portable vacuum source 330 may be sized to fit in the chamber 104 inside of the fluid impermeable barrier 102. In some examples, the portable vacuum source 330 may be sealed in a fluid tight housing or container. The portable vacuum source 330 may apply a vacuum (e.g., suction) in the A section of the conduit 108 effective to suction fluid from the chamber 104. The fluid may travel through the A section to the B section (e.g., through the portable vacuum source 330) and out of the fluid collection device 300 via the B section by flow induced by the vacuum or suction applied by the portable vacuum source 330. For example, the portable vacuum source 330 may include a centrifugal pump and an impeller therein may draw the fluid from the chamber 104 via the inlet 110 and force the fluid out of the chamber 104 via the B section of the conduit 108. Each of the A second and the B section of the conduit 108 may be fluidly coupled (e.g., sealed) to the portable vacuum source 330.

In some examples, the portable vacuum source 330 and the conduit 108 can be integrally formed together (e.g., exhibit single piece construction).

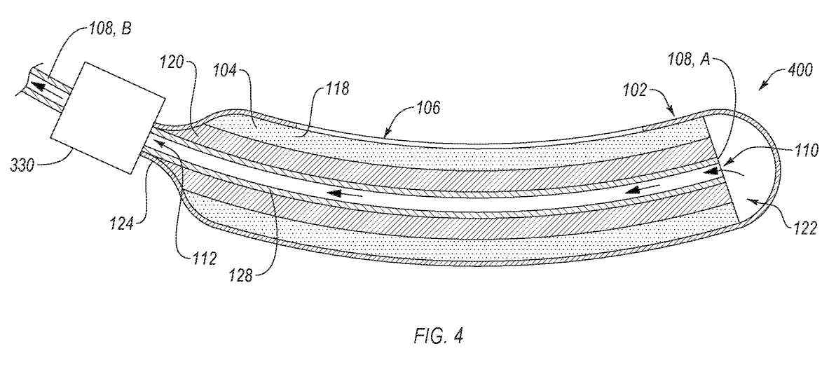

FIG. 4 is a schematic cross-sectional view of a fluid collection device 400, according to an embodiment. The fluid collection device 400 may include the portable vacuum source 330 disposed thereon. Except as otherwise disclosed herein, the fluid collection device 400 can be the same as or substantially similar to the fluid collection device 100 of FIG. 2, in one or more aspects. For example, the fluid collection device 400 can include the fluid impermeable barrier 102 that defines the chamber 104 and the opening 106. The fluid collection device 400 also includes at least one of the fluid permeable membrane 118, the fluid permeable support 120, and the reservoir 122 disposed in the chamber 104. The portable vacuum source 330 may be attached to the fluid collection device 400 at the fluid impermeable barrier 102.

The fluid collection device 400 includes the conduit 108 that is at least partially disposed in the chamber 104. The conduit 108 can include one or more walls that define an inlet 110 and the outlet 112. The inlet 110 enables at least some of the fluids that are present in the chamber 104 to enter the conduit 108. In an example, the conduit 108 can be configured to have the inlet 110 located at, near, or spaced at a gravimetrically low point of the chamber 104. In an example, the conduit 108 can be configured to have the at least one inlet 110 disposed in or adjacent to the reservoir 122.

The conduit 108 can be in fluid communication with the interior region of the chamber 104 via the fluid impermeable barrier 102. As such, the fluid impermeable barrier 102 can define an aperture 124. In an example, as illustrated, the aperture 124 enables the conduit 108 to extend outwardly from the chamber 104 when the conduit 108 is only partially disposed in the chamber 104. In some examples, the conduit 108 may include a plurality of separate sections. For example and as shown, the conduit 108 may include the first section A and the second section B. The first section A may include the inlet 110 extending from the distal end (e.g., first end region), out of the aperture 124, to the portable vacuum source 330 mounted thereto. The portable vacuum source 330 may be mounted to the outer surface of the fluid collection device 400, such as on the fluid impermeable barrier 102. The B section may be attached to and extend from the portable vacuum source 330, such as to a fluid storage container (not shown).

The portable vacuum source 330 may include any of the portable vacuum pumps disclosed herein such as a manual vacuum pump, a diaphragm pump, a centrifugal pump, a displacement pump, a magnetically driven pump, a peristaltic pump, or any pump configured to produce a vacuum. The portable vacuum source 330 may be sized to fit in the chamber 104 inside of the fluid impermeable barrier 102. In some examples, the portable vacuum source 330 may be sealed in a fluid tight housing or container. The portable vacuum source 330 may apply a vacuum (e.g., suction) in the A section of the conduit 108 effective to suction fluid from the chamber 104. The fluid may travel through the A section out of the fluid collection device 400 to the portable vacuum source 330. The fluid may be removed from the portable vacuum source 330 via the B section by flow induced by the vacuum or suction applied by the portable vacuum source 330. For example, the portable vacuum source 330 may include a centrifugal pump and an impeller therein may draw the fluid from the chamber 104 via the inlet 110 and suction the fluid out of the chamber 104 via the portable vacuum source 330 to the B section of the conduit 108. Each of the A section and the B section of the conduit 108 may be fluidly coupled (e.g., sealed) to the portable vacuum source 330. In some examples, the portable vacuum source 330 and the conduit 108 (e.g., one or both of the A section or the B section) can be integrally formed together to exhibit single piece construction. The female fluid collection devices disclosed herein may also include one or more vacuum relief holes in the fluid impermeable barrier 102 to control an amount of suction or vacuum in the chamber 104. The vacuum relief hole may be positioned at any point on the fluid permeable membrane, such as at an intermediate point between the reservoir 122 and the outlet 112. Such vacuum relief holes may allow the chamber 104 to remain substantially at atmospheric pressure to prevent the fluid collection devices from deforming under vacuum force.

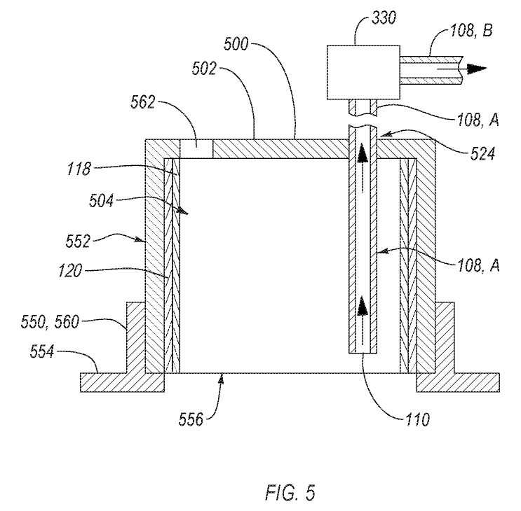

The fluid collection devices shown in FIGS. 2-4 are examples of female fluid collection devices that are configured to collect fluid(s) from females (e.g., collect urine from a female urethra). However, the fluid collection devices, systems, and methods disclosed herein can include male fluid collection devices shaped, sized, and otherwise configured to collect fluid(s) from males (e.g., collect urine from a male urethra). FIGS. 5-7 are schematic cross-sectional views of male fluid collection devices 500, 600, and 700, according to different embodiments.

Referring to FIG. 5, the fluid collection device 500 includes a receptacle 550 and a cup portion 552. The receptacle 550 is sized, shaped, and made of a material to be coupled to skin that surrounds the male urethra and have the male urethra positioned therethrough. For example, the receptacle 550 can include an annular base 554 that defines an opening 556 in the receptacle 550. The annular base 554 is sized and shaped to be positioned around the male urethra (e.g., positioned around and/or over the penis) and the opening 556 can be configured to have the male urethra positioned therethrough. The annular base 554 can also be sized, shaped, made of a material, or otherwise configured to be coupled (e.g., adhesively attached, such as with a hydrogel adhesive) to the skin around the male urethra (e.g., around the penis). In an example, the annular base 554 can exhibit the general shape of the skin surface that the annular base 554 is selected to be coupled with and/or can be flexible thereby allowing the annular base 554 to conform to any shape of the skin surface. The receptacle 550 also defines a hollowed region that is configured to receive (e.g., seal against) the cup portion 552. For example, the receptacle 550 can include a flange 560 that extends upwardly from the annular base 554. The flange 560 may be tall enough to prevent the cup portion 552 from being accidentally removed from the receptacle 550 (e.g., at least 0.5 cm tall, 1 cm tall, at least 2 cm tall, or at least 5 cm tall). In some examples, the annular base 554 is optional. For example, the receptacle 550 may only include the flange 554. In some examples (not shown), the fluid collection device may have a one piece design, with the cup portion 552 and the receptacle 550 being a single piece. In some examples, the receptacle 550 is optional.

The cup portion 552 includes (e.g., may be formed from) a fluid impermeable barrier 502 that is sized and shaped to fit into the hollowed region of the receptacle 550. The cup portion 552 may be shaped to retain a fluid therein. For example, the fluid impermeable barrier 502 may define the cup portion 552, such as forming a substantially tubular (e.g., cylindrical) body having an enclosed end as illustrated in FIG. 5. Accordingly, the cup portion 552 may have a generally cupped shape with a chamber 504 therein. The fluid impermeable barrier 502 may be similar or identical to the fluid impermeable barrier 102, in one or more aspects. The fluid impermeable barrier 502 partially defines the chamber 504. The fluid impermeable barrier 502 may also define an opening 556 extending through the fluid impermeable barrier 502 that is configured to have a male urethra positioned therethrough. The fluid impermeable barrier 502 may also include at least one passageway 562 (e.g., vacuum relief hole) that allows the chamber 504 to remain substantially at atmospheric pressure. The at least one passageway 562 may be located at any point on the cup portion 552, such as near or nearer the opening 556. The cup portion 552 also includes at least a portion of the conduit 108 therein, such as at least partially disposed in the chamber 504. For example, the conduit 108 may extend from the cup portion 552 to a region at least proximate to the opening 556. The region proximate to the opening 556 may be disposed near or on the skin around the male urethra (e.g., on the penis). Accordingly, when a patient lays on their back, fluid (e.g., urine) may aggregate near the opening 556 against the skin of the subject. The fluid may be removed from the chamber 504 via the conduit 108. In some examples, the cup portion 552 of the fluid impermeable barrier 502 may be constructed of a material and/or have a thickness that allows the cup portion 552 to collapse when placed under vacuum, such as to remove air around a penis in the fluid collection device 500 during use. In such examples, the conduit 108 may extend only into the chamber 504 at the aperture 524 (e.g., not through to the area adjacent the opening). In such examples, urine may be collected and removed from the fluid collection device 500 at the end nearest the aperture 524. In such examples, the at least one passageway may be located nearest the opening 556.

The fluid collection device 500 may include the fluid permeable membrane 118. The fluid permeable membrane 118 may be disposed between the fluid impermeable barrier 502 of the cup portion 552 and a penis inserted into the chamber 504. The fluid collection device 500 may include a fluid permeable support 120. The fluid permeable support 120 may be positioned between the cup portion 552 and a penis inserted into the chamber 504, such as between the fluid permeable membrane 118 and the fluid impermeable barrier 502. The sidewalls or the end of the chamber 504 may be covered with one or both the fluid permeable membrane 118 or the fluid permeable support 120.

In some examples, the portable vacuum source 330 may be remotely located from the cup portion 552. In such examples, the conduit 108 may extend out of and away from the cup portion 552 to the portable vacuum source 330. In some examples, the conduit 108 may include one or more sections. For example and as shown, the conduit 108 may include an A section and a B section. The A section may extend from portable vacuum source 330 through the cup portion 552 via the aperture 524 to the region at least proximate to the opening 556. The inlet 110 of the conduit is in fluid communication with the portable vacuum source 330. The outlet (not shown) may be in fluid communication with a fluid storage container (not shown) through the conduit 108 in the direction shown by the arrows. The fluid impermeable barrier 502 may include at least one aperture 524 that is sized and shaped to receive and seal against the conduit 108, such as within the chamber 504. Accordingly, the interior region of the chamber 504 may be in fluid communication with the portable vacuum source 330 via the conduit 108. As the portable vacuum source 330 applies a vacuum/suction in the direction of the arrows in FIG. 5, the fluid in the chamber 504 may be removed through the conduit 108. In some examples, the fluid may be pumped through the portable vacuum source 330 into the B section of the conduit 108. The B section of the conduit may be in fluid communication with a fluid storage container (not shown) into which the fluid may be deposited.

In some examples, the fluid storage container (not shown) may be disposed between the portable vacuum source 330 and the fluid collection device 500. In such examples, the A section of the conduit 108 may extend between the fluid collection device and the fluid storage device, the B section of the conduit 108 may extend between the fluid storage device and the portable vacuum source 330. The A and B sections of the conduit 108 and the fluid storage container may fluidly couple the portable vacuum source 330 to the fluid collection device 500. In such examples, the fluid may be suctioned from the chamber 504 into the inlet 510 and the fluid storage container (not shown) via the vacuum/suction induced by the portable vacuum source 330 through the fluid storage device.

In an example, portions of the chamber 504 may be substantially empty due to the varying sizes and rigidity of the male penis. However, in some examples, the outermost regions of the chamber 504 (e.g., periphery of the interior regions of the cup portion 552) can include a porous material (e.g., one or more of the fluid permeable membrane 118 and fluid permeable support 120, FIG. 2) positioned (e.g., at the end of the cavity) configured to blunt a stream of urine from the male urethra thereby limiting splashing and/or to direct the fluid(s) to a selected region of the chamber 504. Since the chamber 504 is substantially empty (e.g., substantially all of the chamber 504 forms a reservoir), the fluids are likely to pool at a gravimetrically low point of the chamber 504. The gravimetrically low point of the chamber 504 can be at an intersection of the skin of an individual and the fluid collection device 500, a corner formed in the cup portion 552, or another suitable location depending on the orientation of the wearer. The inlet 110 of the conduit 108 can be positioned to be adjacent or proximate to the gravimetrically low point of the chamber 504, such as adjacent to the annular base 554. For example, the inlet 110 may be co-extensive with or offset from the opening 556. In examples, the inlet may be positioned adjacent to the terminal end of the cup portion 552 (e.g., substantially opposite the opening).

During operation, a male using the fluid collection device 500 can discharge fluid(s) (e.g., urine) into the chamber 504. The fluid(s) can pool or otherwise be collected in the chamber 504. At least some of the fluid(s) can enter the interior of the conduit 108 via the inlet 110. The fluid may be drawn out of the fluid collection device 500 via the vacuum/suction provided by the portable vacuum source 330. In some examples, during operation, the passageway 562 may substantially maintain the pressure in the chamber 504 at atmospheric pressure even though fluid is introduced into and subsequently removed from the chamber 504.

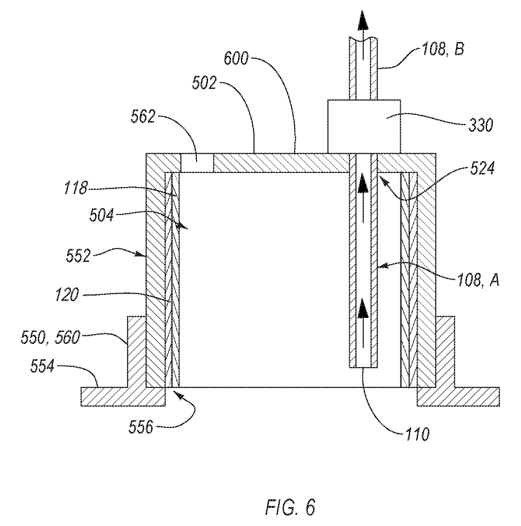

In some examples, the portable vacuum source 330 may be located on the cup portion 552. FIG. 6 illustrates a fluid collection device 600 that, except as otherwise disclosed herein, is the same as or substantially similar to the fluid collection device 500 of FIG. 5, in one or more aspects. For example, the components of the fluid collection device 600 may be identical to the components of the fluid collection device 500 described above. Referring to FIG. 6, the fluid collection device 600 includes the portable vacuum source 330, the receptacle 550, the cup portion 552, and the conduit 108. As shown, the portable vacuum source 330 may be affixed to the fluid collection device 500 such as on the fluid impermeable barrier 102 defining the cup portion 552 or the receptacle 550. In such examples, the B portion of the conduit 108 may extend away from the fluid collection device 500.

The conduit 108 may extend from the chamber 504 of the cup portion 552 to the portable vacuum source 330. In some examples, the conduit 108 may include one or more sections. For example and as shown, the conduit 108 may include an A section and a B section. The A section may extend from portable vacuum source 330 through the cup portion 552 via the aperture 524 to the region at least proximate to the opening 556. The inlet 110 of the conduit is in fluid communication with the portable vacuum source 330. The outlet (not shown) may be in fluid communication with a fluid storage container (not shown) through the conduit 108 in the direction shown by the arrows. The fluid impermeable barrier 502 may include at least one aperture 524 that is sized and shaped to receive and seal against the conduit 108, such as within the chamber 504. Accordingly, the interior region of the chamber 504 may be in fluid communication with the portable vacuum source 330 via the conduit 108. As the portable vacuum source 330 applies a vacuum/suction in the direction of the arrows in FIG. 6, the fluid in the chamber 504 may be removed through the conduit 108. In some examples, the fluid may be pumped through the portable vacuum source 330 into the B section of the conduit 108. The B section of the conduit may be in fluid communication with a fluid storage container (not shown) into which the fluid may be deposited.

In some examples, the portable vacuum source 330 may be disposed between the fluid storage device (not shown) and the fluid collection device 600. In such examples, the A section of the conduit 108 may extend between the fluid collection device 600 and the portable vacuum source 330, the B section of the conduit 108 may extend between the portable vacuum source 330 and the fluid storage container. The A section of the conduit 108 may fluidly couple the portable vacuum source 330 to the fluid collection device 600. In such examples, the fluid may be suctioned from the chamber 504 into the inlet 510 through the portable vacuum source 330 to the fluid storage container (not shown) via the vacuum/suction induced by the portable vacuum source 330. In some examples, the fluid storage container may be located in the chamber 504, on the cup portion 552, or remote from the cup portion 552. In some examples, the fluid storage container (not shown) may be disposed between the portable vacuum source 330 and the fluid collection device 600.

In an example, portions of the chamber 504 of the fluid collection device 600 may be substantially empty or can include a porous material configured to blunt a stream of urine from the male urethra. Since the chamber 504 of the fluid collection device 600 is substantially empty, the fluid(s) are likely to pool at a gravimetrically low point of the chamber 504. The inlet 110 of the conduit 108 can be positioned to be adjacent or proximate to the gravimetrically low point of the chamber 504 of the fluid collection device 600.

During operation, a male using the fluid collection device 600 can discharge fluid(s) (e.g., urine) into the chamber 504. The fluid(s) can pool or otherwise be collected in the chamber 504. At least some of the fluid(s) can enter the interior of the conduit 108 via the inlet 110. The fluid may be drawn out of the fluid collection device 600 via the vacuum/suction provided by the portable vacuum source 330. In some examples, during operation, the passageway 562 may substantially maintain the pressure in the chamber 504 at atmospheric pressure even though fluid is introduced into and subsequently removed from the chamber 504.

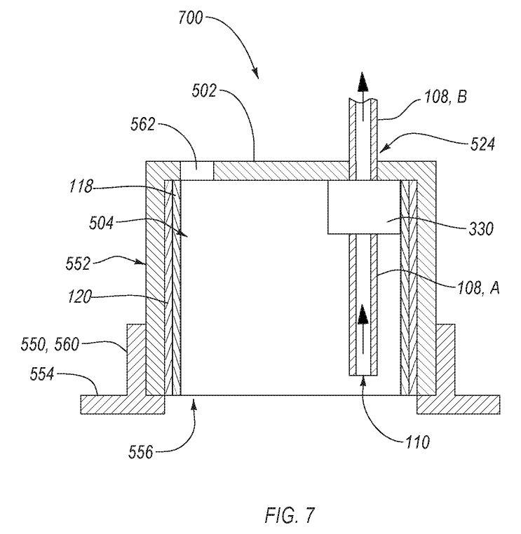

In some examples, the portable vacuum source 330 may be located in the cup portion 552. FIG. 7 illustrates a fluid collection device 700 that, except as otherwise disclosed herein, is the same as or substantially similar to the fluid collection device 500 of FIG. 5 or 600 of FIG. 6 in one or more aspects. For example, the components of the fluid collection device 700 may be identical to the components of the fluid collection device 500 or 600 described above. Referring to FIG. 7, the fluid collection device 700 includes the portable vacuum source 330, the receptacle 550, the cup portion 552, and the conduit 108. As shown, the portable vacuum source 330 may be affixed to the fluid collection device 700 such as on the cup portion 552 or the receptacle 550. In such examples, the B portion of the conduit 108 may extend out of and away from the fluid collection device 700.

The conduit 108 may extend from the chamber 504 of the cup portion 552 to the portable vacuum source 330. In some examples, the conduit 108 may include one or more sections. For example and as shown, the conduit 108 may include an A section and a B section. The A section may extend from portable vacuum source 330 in the cup portion 552 to the region at least proximate to the opening 556. The inlet 110 of the conduit is in fluid communication with the portable vacuum source 330. The outlet (not shown) may be in fluid communication with a fluid storage container (not shown) through the conduit 108 in the direction shown by the arrows. The fluid impermeable barrier 502 may include at least one aperture 524 that is sized and shaped to receive and seal against the conduit 108, such as within the chamber 504. Accordingly, the interior region of the chamber 504 may be in fluid communication with the portable vacuum source 330 via the conduit 108. As the portable vacuum source 330 applies a vacuum/suction in the direction of the arrows in FIG. 7, the fluid in the chamber 504 may be removed through the conduit 108. In some examples, the fluid may be pumped through the portable vacuum source 330 into the B section of the conduit 108. The B section of the conduit may extend out of the aperture 524 to a fluid storage container (not shown) fluidly coupled thereto, into which the fluid may be deposited.

In some examples, the portable vacuum source 330 may be disposed between the fluid storage device (not shown) and the fluid collection device 700. In such examples, the A section of the conduit 108 may extend between the chamber 504 of the fluid collection device 700 and the portable vacuum source 330, the B section of the conduit 108 may extend between the portable vacuum source 330 and the fluid storage container. The A section of the conduit 108 may fluidly couple the portable vacuum source 330 to the fluid collection device 700. In such examples, the fluid may be suctioned from the chamber 504 into the inlet 510 through the portable vacuum source 330 to the fluid storage container (not shown) via the vacuum/suction induced by the portable vacuum source 330. In some examples, the fluid storage container may be located in the chamber 504, on the cup portion 552, or remote from the cup portion 552. In some examples, the fluid storage container (not shown) may be disposed between the portable vacuum source 330 and the fluid collection device 700.

In an example, portions of the chamber 504 of the fluid collection device 700 may be substantially empty due to the varying sizes and rigidity of the male penis. However, in some examples, the outermost regions of the chamber 504 can include a porous material configured to blunt a stream of urine from the male urethra. Since the chamber 504 is substantially empty, the fluids are likely to pool at a gravimetrically low point of the chamber 504. The inlet 110 of the conduit 108 can be positioned to be adjacent or proximate to the gravimetrically low point of the chamber 504 of the fluid collection device 700.

During operation, a male using the fluid collection device 700 can discharge fluid(s) (e.g., urine) into the chamber 504. The fluid(s) can pool or otherwise be collected in the chamber 504. At least some of the fluid(s) can enter the interior of the conduit 108 via the inlet 110. The fluid may be drawn out of the fluid collection device 700 via the vacuum/suction provided by the portable vacuum source 330. In some examples, during operation, the passageway 562 may substantially maintain the pressure in the chamber 504 at atmospheric pressure even though fluid is introduced into and subsequently removed from the chamber 504.

In any of the examples disclosed herein, the conduits 108 may include or be operably coupled to a flow meter (not shown) to measure the flow of fluid(s) therein, one or more securement devices (e.g., a STATLOCK® securement device, not shown) or fittings to secure the conduit 108 to one or more components of the systems or devices disclosed herein (e.g., portable vacuum source or fluid storage container), or one or more valves to control the flow of fluid(s) in the systems and devices herein. In any of the examples disclosed herein, the conduits 108 or other portions of the devices and systems may include a port for inserting one or more testing devices to determine if the collected urine indicates an onset of an infection (e.g., urinary tract infection or kidney infection). For example, one or more testing strips or sticks may be inserted through a port in the conduit 108.

In an example, at least one of portion of the conduit 108 of the fluid collection devices or systems herein can be formed of an at least partially opaque material which can obscure the fluids that are present therein. For example, the B section of the conduits 108 disclosed herein may be formed of an opaque material or translucent material while the A section may be formed of a transparent material or translucent material. In some examples, the B section may include transparent or translucent material. Unlike the opaque or nearly opaque material, the translucent material allows a user of the devices and systems herein to visually identify fluid(s) or issues that are inhibiting the flow of fluid(s) within the conduit 108.

In any of the example, systems or devices disclosed herein, the system of fluid collection device may include moisture sensors (not shown) disposed inside of the chamber of the fluid collection device. In such examples, the moisture sensor may be operably coupled to a controller or directly to the portable vacuum source, and may provide electrical signals indicating that moisture is or is not detected in one or more portions of the chamber. The moisture sensor(s) may provide an indication that moisture is present, and responsive thereto, the controller or portable vacuum device may direct the initiation of suction to the chamber to remove the fluid therefrom. Suitable moisture sensors may include capacitance sensors, volumetric sensors, potential sensors, resistance sensors, frequency domain reflectometry sensors, time domain reflectometry sensors, or any other suitable moisture sensor. In practice, the moisture sensors may detect moisture in the chamber and may provide a signal to the controller or portable vacuum source to activate the portable suction device.

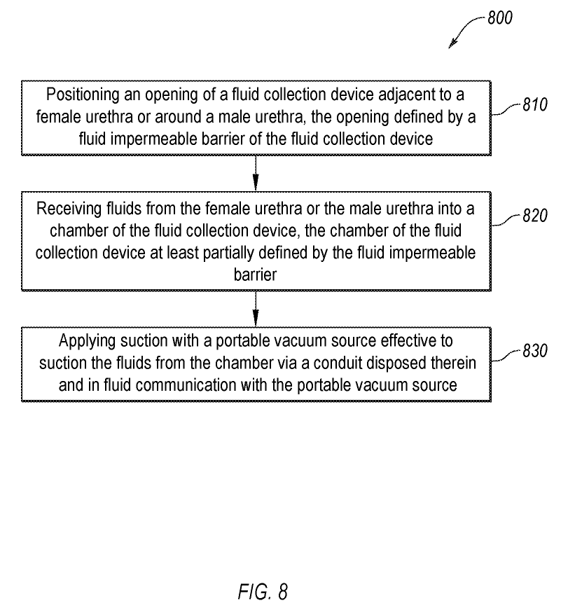

FIG. 8 is a flow diagram of a method 800 to use any of the fluid collection devices and/or fluid collection systems disclosed herein, according to an example. The method 800 can include act 810, which recites “positioning an opening of a fluid collection device adjacent to a female urethra or around a male urethra, the opening defined by a fluid impermeable barrier of the fluid collection device.” Act 810 may be followed by act 820, which recites “receiving fluid from the female urethra or the male urethra into a chamber of the fluid collection device, the chamber of the fluid collection device at least partially defined by the fluid impermeable barrier.” Act 820 may be followed by act 830, which recites “applying suction with a portable vacuum source effective to suction the fluid from the chamber via a conduit disposed therein and in fluid communication with the portable vacuum source.”

Acts 810, 820, 830 of the method 800 are for illustrative purposes. For example, the act 810, 820, 830 of the method 800 can be performed in different orders, split into multiple acts, modified, supplemented, or combined. In an example, one or more of the acts 810, 820, 830 of the method 800 can be omitted from the method 800. Any of the acts 810, 820, or 830 can include using any of the fluid collection devices or systems disclosed herein.

Act 810 recites “positioning an opening of a fluid collection device adjacent to a female urethra or around a male urethra, the opening defined by a fluid impermeable barrier of the fluid collection device.” In some examples, act 810 can include positioning the opening of a female fluid collection device such that the fluid permeable membrane of the female fluid collection device abuts or is positioned proximate to the female urethra. In some examples, act 810 can include positioned a receptacle of a male fluid collection device around (e.g., over) the male urethra such that the male urethra is positioned in the receptacle. In such an example, act 810 can include positioning a cup portion of the male fluid collection device in a hollowed region of the receptacle such that the male urethra is positioned through an opening of the cup portion of the male fluid collection device and into the interior cavity of the male fluid collection device. In some examples, the act 810 may include positioning a penis within the fluid collection device, such as in the chamber thereof. In some examples, positioning an opening of a fluid collection device adjacent to a female urethra or around a male urethra may include positioning the opening over the female urethra, such as positioning a longitudinally extending opening of the fluid collection device over the female urethra.

Act 820 recites “receiving fluid from the female urethra or the male urethra into a chamber of the fluid collection device, the chamber of the fluid collection device at least partially defined by the fluid impermeable barrier.” For example, act 820 can include wicking the fluid(s) away from the opening using a fluid permeable membrane and a fluid permeable support. In some examples, act 820 can include receiving the fluid(s) into the chamber of the cup portion of the male fluid collection device. In either example, act 820 can include flowing the fluid towards a portion of the chamber that is in fluid communication with an inlet of a conduit in fluid communication a vacuum source. For instance, act 820 can include flowing the fluid(s) to a substantially unoccupied portion of the chamber (e.g., a reservoir), to a gravimetrically low point of the chamber, etc. In some examples, receiving fluid(s) from the female urethra or the male urethra into a chamber of the fluid collection device may include wicking the fluid (e.g., urine) into the chamber via the fluid permeable membrane and fluid permeable support of the fluid collection device. For example, wicking the fluid into the chamber via the fluid permeable membrane and fluid permeable support may include wicking urine into a reservoir in the fluid collection device.

Act 830 recites, “applying suction with a portable vacuum source effective to suction the fluid(s) from the chamber via a conduit disposed therein and in fluid communication with the portable vacuum source.” In some examples, applying suction with a portable vacuum source effective to suction the fluid(s) from the chamber via a conduit disposed therein and in fluid communication with the portable vacuum source can include using any of the portable vacuum sources disclosed herein. In an example, act 830 can include activating the portable vacuum source (e.g., portable suction device) in fluid communication with the inlet of the conduit in the fluid collection device. In some examples, activating the portable vacuum source in fluid communication with the inlet of the conduit in the fluid collection device can include supplying power to the portable vacuum source by one or more of flipping an on/off switch, pressing a button, plugging the portable vacuum source into a power outlet, putting batteries into the portable vacuum source, etc. In some examples, the portable vacuum source may include a hand operated vacuum pump and applying suction with a portable vacuum source may include manually operating the hand operated vacuum pump effective to suction the fluid(s) from the chamber via the conduit disposed therein that is in fluid communication with the portable vacuum source.

In some examples, applying suction with a portable vacuum source effective to suction the fluid(s) from the chamber via a conduit disposed therein and in fluid communication with the portable vacuum source can be effective to remove at least some fluid (e.g., urine) from the chamber (e.g., interior region) of the fluid collection device. In some examples, applying suction with a portable vacuum source effective to suction the fluid(s) from the chamber via a conduit disposed therein and in fluid communication with the portable vacuum source can be effective to transfer at least some of the fluid from the chamber of the fluid collection device to a fluid storage container (e.g., a bottle or bag). In some examples, applying suction with a portable vacuum source effective to suction the fluid(s) from the chamber may include removing fluid from one or more of a reservoir, fluid permeable support, or fluid permeable membrane of the fluid collection device.

In some examples, the portable vacuum source (e.g., suction device) may be disposed on or within the fluid collection device and applying suction with the portable vacuum source may include activating the portable vacuum source. In some examples, the portable vacuum source may be spaced from the fluid collection device and applying suction with the portable vacuum source may include activating the portable vacuum source.

In some examples, applying suction with a portable vacuum source effective to suction the fluid(s) from the chamber via a conduit disposed therein and in fluid communication with the portable vacuum source may include detecting moisture in the chamber (e.g., via one or more moisture sensors) and responsive thereto, activating the portable vacuum source to provide suction in the chamber. The control of the portable vacuum source responsive to the signals indicating that moisture or a level thereof is present in the chamber can be automatic, such as via a controller (e.g., computer programmed to perform the operation), or may merely provide an indication that a level of moisture is present that may necessitate removal of fluid from the chamber of the fluid collection device. In the latter case, a user may receive the indication (e.g., from the controller) and activate the portable vacuum pump manually.

In an example, the method 800 can include collecting the fluid(s) that are removed from the fluid collection device, such as into a fluid storage container that is spaced from the fluid collection device and in fluid communication with the conduit. The fluid storage container can include any of the fluid storage containers disclosed herein.

While various aspects and embodiments have been disclosed herein, other aspects and embodiments are contemplated. The various aspects and embodiment disclosed herein are for purposes of illustration and are not intended to be limiting.

We claim:

1. A urine collection system, comprising:
a fluid storage container configured to hold a fluid;
a wearable urine collection device in fluid communication with the fluid storage container, the urine collection device including:
a fluid impermeable barrier at least partially defining a chamber, the fluid impermeable barrier also defining an opening extending therethrough, the opening configured to be positioned adjacent to a female urethra or have a male urethra positioned therethrough; and
a conduit including an inlet and an outlet, the inlet being positioned within the urine collection device and the outlet is configured to be in fluid communication with the fluid storage container; and
a portable vacuum source in fluid communication with the fluid storage container, wherein the portable vacuum source is disposed within the fluid impermeable barrier of the urine collection device and the portable vacuum source is configured to draw liquid from the urine collection device;
wherein the conduit includes a first portion extending and connected to the portable vacuum source from outside of the fluid impermeable barrier and a second portion extending from and connected to the portable vacuum source within the fluid impermeable barrier with the inlet positioned in the chamber.
